(19) United States
(12) Patent Application Publication
(10) Pub. No.: US 2012/0276648 A1
(43) Pub. Date: Nov. 1, 2012

(54) ELECTROSTATICALLY STABILIZED METAL SULFIDE NANOPARTICLES FOR COLORIMETRIC MEASUREMENT OF HYDROGEN SULFIDE

(75) Inventors: Ronald E.G. van Hal, Watertown, MA (US); Jimmy Lawrence, Amherst, MA (US); Jane T. Lam, Randolph, MA (US)

(73) Assignee: Schlumberger Technology Corporation, Cambridge, MA (US)

(21) Appl. No.: 13/097,380

(22) Filed: Apr. 29, 2011

(51) Int. Cl. G01N 33/00

(52) U.S. Cl. 436/119

(57) ABSTRACT

Methods and related apparatuses and mixtures are described for spectroscopic detection of hydrogen sulfide in a fluid, for example a formation fluid downhole. A reagent mixture is combined with the fluid. The reagent mixture includes metal ions for reacting with hydrogen sulfide forming a metal sulfide, and a solvent that stabilizes the metal sulfide nanoparticles and assist in preventing precipitation by electrostatic stabilization. The solvent includes a property having a density above 1 kg/l. Further, dissolving the metal ions into the solvent to create the reagent mixture, and mixing the reagent mixture with the hydrogen sulfide sample in a formation. So, the metal ions of the reagent mixture react with the hydrogen sulfide sample to form the metal sulfide nanoparticles, resulting in the metal sulfide nanoparticles having properties with a density from 1 kg/l to about 8 kg/l.

### FIELD

The disclosed subject matter is generally related to detection and sensing of properties of fluids, such as formation fluids. More particularly, this patent specification relates to spectroscopic detection of substances such as Hydrogen Sulfide using reagent mixtures, the reagent mixtures include: metal ions for reacting with the hydrogen sulfide that forms a metal sulfide, along with a solvent that stabilizes the metal sulfide nanoparticles and assist in preventing precipitation by electrostatic stabilization.

### BACKGROUND

Hydrogen sulfide (H₂S) occurs extensively in a number of subsurface hydrocarbon reservoirs under anaerobic conditions. The presence of hydrogen sulfide is highly corrosive to casing, tubing, and other metallic and polymeric tools, an effect that is considerably accelerated by low pH and the presence of carbon dioxide. This has a significant impact on the overall hydrocarbon recovery processes, during which materials selection and corrosion control are of great importance. Additionally, H₂S is hazardous to humans even at minute concentration levels (for example, about 100 ppm).

The H₂S content of reservoir fluids can be determined from samples collected by fluid sampling tools such as wireline fluid sampling tools or other sampling tools. Fluid samples are usually collected in metal containers, which are able to maintain the pressures at which the samples were collected. However, a problem associated with sampling fluids containing hydrogen sulfide is partial loss of the gas by reaction of the metal components, particularly those made from iron-based metals. The hydrogen sulfide gas readily forms non-volatile and insoluble metal sulfides by reaction with many metals and metal oxides, and analysis of the fluid samples can therefore give an underestimate of the true sulfide content.

Moreover, determining H₂S concentration in downhole has also been difficult especially at low concentrations due to H₂S scavenging occurring during the time when the samples are taken and brought for analysis. Thus, it is critically important for oil companies to assess the sulfur content of the reservoir fluid before they make a large investment to the field development. However, detecting sulfur in the early stage of the exploration is not easy or straightforward. It is noted, lab measurements almost always underestimate the H₂S concentration due to scavenging by a formation sampling tool and sampling bottle, as noted above. Detecting sulfur content in heavy crude compounds is done by elemental analysis in a laboratory. While scavenging is not generally an issue for sulfurs in heavy compounds, the long lead-time, at least a month, more often much longer, is not suited for quick decision making. Therefore, in-situ, real time gas detection, particularly hydrogen sulfide is important for downhole fluid analysis

As a result, the in situ detection and measurement of hydrogen sulfide is widely regarded as a critical parameter needed for well completion and production strategies. Due to the high chemical reactivity of sulfide species, various detection strategies including spectroscopy, electrochemistry, chromatography and combinations thereof have been proposed. For example, see Wardencki, W. J. “Problems with the determination of environmental sulphur compounds by gas chromatography” Journal of Chromatography A, Vol 793, 1 (1998). U.S. Pat. No. 6,939,717B2 describes feasible electrochemical and optical methodologies and embodiments aimed at downhole detection of hydrogen sulfide.

### SUMMARY

The present disclosed subject matter relates to a reagent mixture for use in spectroscopic detection of hydrogen sulfide. The reagent mixture comprising: (a) metal ions dissolved in a fluid, the fluid is one of a ionic liquid or a polar organic solvent for reacting with a hydrogen sulfide sample thereby forming a metal sulfide nanoparticles. Nanoparticles can be suspended if the interaction of the particle surface with the solvent is strong enough to overcome density differences. Wherein at least one polar organic solvent can include two or more properties with a density above 1 kg/l and a electrical dipole moment above 2 Debye units can have this ability. The ionic liquid and the at least one polar organic solvent can provide for stabilizing the metal sulfide nanoparticles and can be used for preventing precipitation by electrostatic stabilization; (b) dissolving the metal ions into one of the ionic liquid or the polar organic solvent to create the reagent mixture; (c) mixing the reagent mixture with the hydrogen sulfide sample in a formation under downhole conditions at a temperature above 100 Celsius and at a pressure above 100 psi, and wherein the metal ions of the reagent mixture react with the hydrogen sulfide sample to form the metal sulfide nanoparticles, resulting in the metal sulfide nanoparticles having an optical signature within the range from 200 nm to about 900 nm.

According to aspects of the subject matter disclosed, wherein the metal ions can be from a soft metal group consisting of cadmium, mercury, silver, gold, palladium, rhodium, ruthenium, osmium, iridium, platinum or thallium. Further, the metal ions can be from the intermediate metal group consisting of manganese, iron, cobalt, nickel, copper, zinc, molybdenum, technetium, indium, tantalum, tungsten, rhenium, lead or bismuth. The method further comprises a chelating ligand to dissolve the metal ions. It is possible a ratio of the chelating ligand to the metal ion can be between about 0 to 2.

According to aspects of the subject matter disclosed, the polar organic solvent can be from an alkanolamines group including triethanolamine, methyldiethanolamine or some combination thereof. Further, the polar organic solvent can be dimethyl sulfoxide (DMSO).

According to aspects of the subject matter disclosed, the spectroscopy can be used for the detection of the hydrogen sulfide after the homogenous reagent mixture is exposed to hydrogen sulfide sample to form the metal sulfide nanoparticles. The metal ions can be cadmium. It is possible that in detecting hydrogen sulfide in the formation fluid can be under downhole conditions at sustained temperatures of one of 100 degree Celsius or more, 200 degree Celsius or more or 250 degree Celsius or more.

The ionic liquid can be from the group consisting of one of ammonium based, phosphonium based or imidazolium based cations. It is possible the ionic liquid contains a dication.

In accordance with another embodiment of the disclosed subject matter, a method for use in spectroscopic detection of hydrogen sulfide in a formation fluid while in a formation. The method includes the method comprising: (a) obtaining metal ions to be dissolved in a fluid, the fluid is one of a ionic liquid or a polar organic solvent for reacting with a hydrogen sulfide sample thereby forming a metal sulfide nanoparticles, the polar organic solvent includes two or more properties having a density above 1 kg/l and a electrical dipole moment above 2 Debye units, wherein the ionic liquid and the polar organic solvent provide for stabilizing the metal sulfide nanoparticles and for preventing precipitation by electrostatic stabilization; (b) combining the metal ions into one of the ionic liquid or the polar organic solvent to create a homogenous reagent mixture; (c) mixing the homogenous reagent mixture with the hydrogen sulfide sample in the formation under downhole conditions at a temperature above 100 Celsius and at a pressure above 200 psi, wherein the metal ions of the homogenous reagent mixture react with the hydrogen sulfide to form the metal sulfide nanoparticles, resulting in the metal sulfide nanoparticles having an optical signature within the range from 200 nm to about 900 nm; and (d) spectroscopically interrogating the homogenous reagent mixture and formation fluid so as to detect the presence of the metal sulfide thereby indicating the presence of hydrogen sulfide in the formation fluid.

According to aspects of the subject matter disclosed, the spectroscopy interrogating can include measuring the optical property such as one of an optical density of the metal sulfide nanoparticles or a fluorescence of the metal sulfide nanoparticles, so a quantity of hydrogen sulfide in the formation fluid is capable of being measured while under downhole conditions. The spectroscopy interrogating can include before mixing, measuring the optical property such as the optical density or the fluorescence of one of the homogenous reagent mixture, the formation fluid or both. The combining can further comprise introducing the homogenous reagent mixture into a downhole flowline containing the formation fluid and the spectroscopically interrogating further comprises interrogating through an optical window in the flowline downstream from the location of introduction of the detection mixture. The combining can further comprise introducing the formation fluid into a container containing the homogenous reagent mixture, and the interrogating further comprises interrogating through an optical window in a wall of the container while in a subterranean environment. The combining can further comprise mechanically stirring the homogenous reagent mixture and the formation fluid in the container to shorten a rate of time used to carry out the interrogating. The combining can further comprise introducing the formation fluid into a container containing the homogenous reagent mixture, and introducing the combined homogenous reagent mixture and the formation fluid from the container into a downhole flowline, and spectroscopically interrogating through an optical window in a wall of the flowline. It is possible to further comprise adding chelating ligands to the homogenous reagent mixture for sustaining thermal endurance of the homogenous reagent mixture under downhole conditions, wherein the metal ions are cadmium, and the spectroscopically interrogating is through an optical window in a wall of the flowline while in a subterranean environment. The method of detecting hydrogen sulfide in the formation fluid can be under downhole conditions is at sustained temperatures of one of 100 degree Celsius or more, 200 degree Celsius or more or 250 degree Celsius or more. It is possible the spectroscopically interrogating of the homogenous reagent mixture and formation fluid can be completed on a surface of the earth.

In accordance with another embodiment of the disclosed subject matter, a reagent mixture for use in spectroscopic detection of hydrogen sulfide. The reagent mixture comprising: (a) metal ions dissolved in a ionic liquid for reacting with a hydrogen sulfide sample thereby forming a metal sulfide nanoparticles, wherein the ionic liquid includes at least one positive ion and at least one negative ion, that provides stabilizing the metal sulfide nanoparticles and for preventing precipitation by electrostatic stabilization; (b) dissolving the metal ions into the ionic liquid to create the reagent mixture; (c) mixing the reagent mixture with the hydrogen sulfide sample in a formation under downhole conditions at a temperature above 150 Celsius and at a pressure above 150 psi, and wherein the metal ions of the reagent mixture react with the hydrogen sulfide sample to form the metal sulfide nanoparticles, resulting in the metal sulfide nanoparticles having properties with an optical signature within the range from 200 nm to about 900 nm and the metal sulfide nanoparticles is a nanoparticle kept in suspension to form a homogenous reagent mixture.

Further features and advantages of the disclosed subject matter will become more readily apparent from the following detailed description when taken in conjunction with the accompanying Drawing.

### BRIEF DESCRIPTION OF THE DRAWINGS

The present disclosed subject matter is further described in the detailed description which follows, in reference to the noted plurality of drawings by way of non-limiting examples of exemplary embodiments of the present disclosed subject matter, in which like reference numerals represent similar parts throughout the several views of the drawings, and wherein:

FIG. 1 shows a prior art schematic diagram showing a downhole/borehole tool with an sampling port;

FIG. 2 shows a prior art schematic diagram of stabilized nanoparticles by electrostatic repulsion, according to the disclosed subject matter;

FIG. 3 shows a prior art schematic diagram of stabilized nanoparticles by steric barrier, according to the disclosed subject matter;

FIG. 4 shows a prior art schematic diagram of stabilized nanoparticles by electrosteric interactions, according to the disclosed subject matter;

FIG. 5 shows the determination of the absorbance threshold wavelength of cadmium sulfide formed in methyldiethanolamine (MDEA) at 100° C., wherein the threshold wavelength is the wavelength were both black lines cross, according to the disclosed subject matter;

FIG. 6 shows a prior art theoretical threshold of absorbance for various metal sulfide particles according to the “effective mass model of quantum confinement,” according to the disclosed subject matter;

FIG. 7 shows the absorbance spectra of various concentrations of cadmium sulfide at room temperature. The cadmium sulfide is formed in a reaction of 4 mM cadmium sulfate in methyldiethanolamine (MDEA) with sulfide at 100° C., according to the disclosed subject matter;

FIG. 8 shows the absorbance as function of the cadmium sulfide concentration at various wavelengths: wherein the absorbance at a certain wavelength is the absorbance at this wavelength minus the absorbance at 550 nm; and the cadmium sulfide is formed in a reaction of 4 mM cadmium sulfate in methyldiethanolamine (MDEA) with sulfide at 100° C., according to the disclosed subject matter;

FIG. 9 shows the absorbance spectra of various concentrations of cadmium sulfide at room temperature, wherein the cadmium sulfide is formed in a reaction of 4 mM cadmium sulfate and 8 mM nitrilotriacetic acid (NTA) in triethanolamine (TEA) with sulfide at 150° C., according to the disclosed subject matter;

FIG. 10 shows the absorbance as function of the cadmium sulfide concentration at various wavelengths: wherein the absorbance at a certain wavelength is the absorbance at this wavelength minus the absorbance at 550 nm; and the cadmium sulfide is formed in a reaction of 4 mM cadmium sulfate and 8 mM nitrilotriacetic acid (NTA) in triethanolamine (TEA) with sulfide at 150° C., according to the disclosed subject matter;

FIG. 11 shows the absorbance spectra of various concentrations of bismuth sulfide, wherein the bismuth sulfide is formed in a reaction of 2 mM bismuth nitrate with and 4 mM nitrilotriacetic acid (NTA) in triethanolamine (TEA) with sulfide at room temperature, according to the disclosed subject matter; and

FIG. 12 shows absorbance curves of triethanolamine (TEA) after 25 hrs exposure to 150° C. Experiments were performed in a closed vessel. Full means that the vessel is filled almost completely 42 ml TEA and 5 ml air, half full means 20 ml TEA and 27 ml air, full water added means 41 ml TEA, 1 ml 0.2 M CdSO4 in water and 5 ml air, according to the disclosed subject matter.

### DETAILED DESCRIPTION OF THE PREFERRED EMBODIMENTS

The particulars shown herein are by way of example and for purposes of illustrative discussion of the embodiments of the present disclosed subject matter only and are presented in the cause of providing what is believed to be the most useful and readily understood description of the principles and conceptual aspects of the present disclosed subject matter. In this regard, no attempt is made to show structural details of the present disclosed subject matter in more detail than is necessary for the fundamental understanding of the present disclosed subject matter, the description taken with the drawings making apparent to those skilled in the art how the several forms of the present disclosed subject matter may be embodied in practice. Further, like reference numbers and designations in the various drawings indicated like elements.

The present disclosed subject matter relates to a mixture that is provided for use in spectroscopic detection of hydrogen sulfide in a fluid, for example a formation fluid downhole. A reagent mixture is combined with the fluid. The reagent mixture includes metal ions for reacting with hydrogen sulfide forming a metal sulfide, and a solvent that stabilizes the metal sulfide nanoparticles and assist in preventing precipitation by electrostatic stabilization. Nanoparticles are defined as particles with at least one dimension sized between 1 and 1000 nanometers. The solvent includes properties having a density above 1 kg/l. Further, the method includes: dissolving the metal ions into the solvent to create the reagent mixture; and mixing the reagent mixture with the hydrogen sulfide sample in a formation under downhole conditions at a temperature above 50 Celsius and at a pressure above 100 psi, wherein the metal ions of the reagent mixture react with the hydrogen sulfide sample to form the metal sulfide nanoparticles, resulting in the metal sulfide nanoparticles having properties with a density from 1 kg/l to about 8 kg/l and an optical signature within the range from 200 nm to about 900 nm.

It is noted that hydrogen sulfide occurs in a large number of subsurface hydrocarbon reservoirs under anaerobic conditions. Further, hydrogen sulfide is highly corrosive to casing, tubing and other metallic tools. This effect is accelerated by low pH and/or the presence of carbon dioxide. Knowledge of the hydrogen sulfide concentration is of great importance for material selection, corrosion control and production strategy. Hydrogen sulfide is scavenged by metals, polymers and unsaturated hydrocarbons. As a result, the in situ (downhole) detection of hydrogen sulfide is regarded as a critical parameter needed for well completion and production strategies. Furthermore, there is a need for an automated system to detect hydrogen sulfide at the well site during well testing

It has been found that a difficulty in hydrogen sulfide (H₂S) detection exits due to the rapid precipitation of the metal sulfides when the H₂S reacts with metal ions, as note above. Since the metal sulfide very quickly precipitates out of the detection solution, its optical detection is often very difficult or impractical. In U.S. 2009/0107667 the inventors try to solve this problem by using electrostatic stabilization with an overwhelming amount of silica nanoparticles in an alkaline aqueous medium. The metal sulfides are clustered on the surface of the silica nanoparticles preventing significant aggregation and precipitation of the metal sulfides. However, the electrostatic effect is highly sensitive to variations in the ionic strength and pH of the suspension. The electrostatic effect becomes less effective at small increases in the ion concentration. Furthermore, the silica suspension tends to form a gel when small amounts of water evaporate, when the ionic strength increases due to contact with salts or salt water and when the pH of the solution is changed by more than 2 pH units. These events are impossible to prevent under downhole conditions where the suspension is exposed to sour gases (H₂S and CO₂), saline water and high temperatures. Gel formation in the flow line will cause the clogging of the flow line.

According to the disclosed subject matter, the metal ions dissolved in a polar high density organic solvent react with hydrogen sulfide to form metal sulfide nanoparticles. The strong electrostatic interaction between the polar solvent and the nanoparticles limits the growth of the nanoparticles. Furthermore, the electrostatic interaction delays the coagulation and subsequent precipitation of the metal sulfide nanoparticles. Addition of an appropriate chelating agent further improves the solubility of the metal sulfide nanoparticles. It is presumed that the chelating agent has a stronger electrostatic interaction than the polar solvent and therefore is the main stabilizer for the nanoparticles. The electrostatically stabilized nanoparticles can be used for the development of a hydrogen sulfide sensor. The optical absorbance of the metal sulfide nanoparticles is directly related to the hydrogen sulfide concentration

According to the disclosed subject matter, it is possible for a specific detection and measurement strategy for hydrogen sulfide is based on the colorimetric features of metal sulfide nanoparticles formed by the reaction of metal ions with sulfide and electrostatically stabilized by a polar organic solvent. For example, solvents used according to the disclosed subject matter, by non-limiting example can be from an alkanolamines group that includes triethanolamine, methyldiethanolamine or some combination thereof. In particular, the solvent can be dimethyl sulfoxide (DMSO). Further still, the aspect of coupling to an optical sensor can be provided for a viable means under downhole conditions and for well site detection of hydrogen sulfide. The Life Fluid Analyser in Schlumberger's openhole logging tool Modular Formation Dynamics Tester (MDT) is an example of an existing downhole optical sensor. Thus, it is possible that systems having the same functionality can be designed for well site applications.

As noted above, the subject matter disclosed relates to methods and devices (or apparatuses) mixing a first fluid such as a reagent mixture with a second fluid such as formation fluid in a downhole environment, wherein at least one embodiment includes the reagent mixture as a liquid and the formation fluid as a single or multiphase fluid. For example, the mixing process will likely be in a tool such as a downhole tool, but other possible devices may be considered. Further, the subject matter disclosed provides many advantages, by non-limiting example, an advantage of mixing downhole fluids effectively in downhole tools. Formation gas or formation liquid can be transferred in to a sample bottle (MPSR) in Schlumberger MRMS Module of the Modular Dynamics Tester (MDT). Another possible advantage, among the many advantages, is that the methods and devices can improve the surface area available for mixing of two fluids (gas-liquid, liquid-liquid, liquid-gas) in a bottle. It is noted that a bottle can be considered a cavity, chamber or any device able to hold fluids.

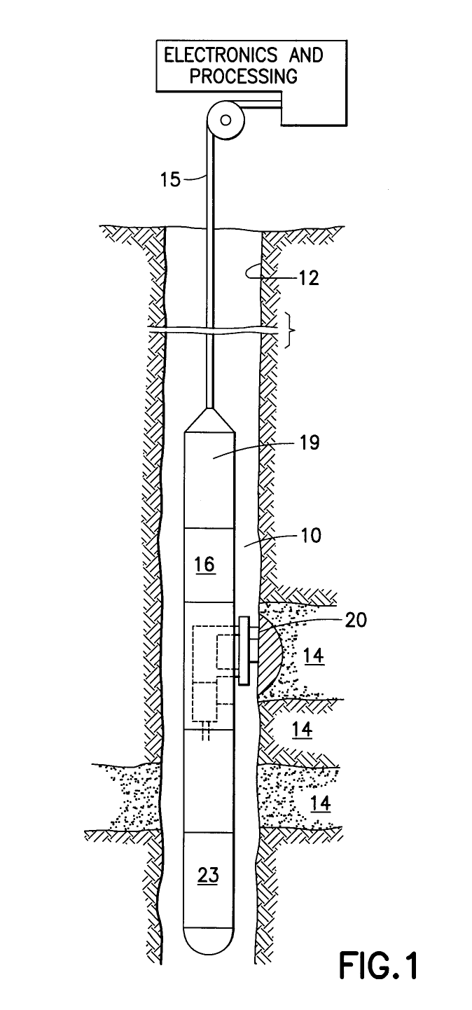

Regarding the downhole tools and methods which expedite the sampling of formation hydrocarbons, the downhole tools, i.e., sampling tools, are utilized to carry downhole the mixing device(s) of the subject matter disclosed in this application. By way of example and not limitation, tools such as the previously described MDT tool of Schlumberger (see, e.g., previously incorporated U.S. Pat. No. 3,859,851 to Urbanosky, and U.S. Pat. No. 4,860,581 to Zimmerman et al.) with or without OFA, CFA or LFA module (see, e.g., previously incorporated U.S. Pat. No. 4,994,671 to Safinya et al., U.S. Pat. No. 5,266,800 to Mullin, U.S. Pat. No. 5,939,717 to Mullins), or the CHDT tool (see, e.g., previously incorporated “Formation Testing and Sampling through Casing”, Oilfield Review, Spring 2002) may be utilized. An example of a tool having the basic elements to implement the invention is seen in schematic in FIG. 1.

FIG. 1 shows a borehole logging tool 10 for testing earth formations and optionally analyzing the composition of fluids from the formation 14 in accord with invention is seen. As illustrated, the tool 10 is suspended in the borehole 12 from the lower end of a typical multiconductor cable 15 that is spooled in the usual fashion on a suitable winch (not shown) on the formation surface. On the surface, the cable 15 is electrically connected to an electrical control system 18. The tool 10 includes an elongated body 19 which encloses the downhole portion of the tool control system 16. The elongated body 19 carries a probe 20 and an anchoring member 21 and/or packers (not shown in FIG. 1). The probe 20 is preferably selectively extendible as is the anchoring member 21 and they are respectively arranged on opposite sides of the body. The probe 20 is equipped for selectively sealing off or isolating selected portions of the wall of borehole 12 such that pressure or fluid communication with the adjacent earth formation is established. Also included with tool 10 is a fluid collecting chamber block 23.

Various other methods to synthesize stable nanocrystals for quantum dot applications may be applicable regarding the stability and aggregation issue, but bring forth issues that cannot be overcome. The differences between quantum dot synthesis and the subject matter disclosed in this application, by non-limiting example, are that the products need to: 1) survive long exposure to high temperature while maintaining its stability; 2) provide an understanding as to how the reagents are reacted with sulfide relating to variables such as temperature, adding time, etc; provide an understanding as to the sulfide amount used in the reaction; 3) provide an understanding to the capping agents employed as to how they maintain stability at wide temperature range.

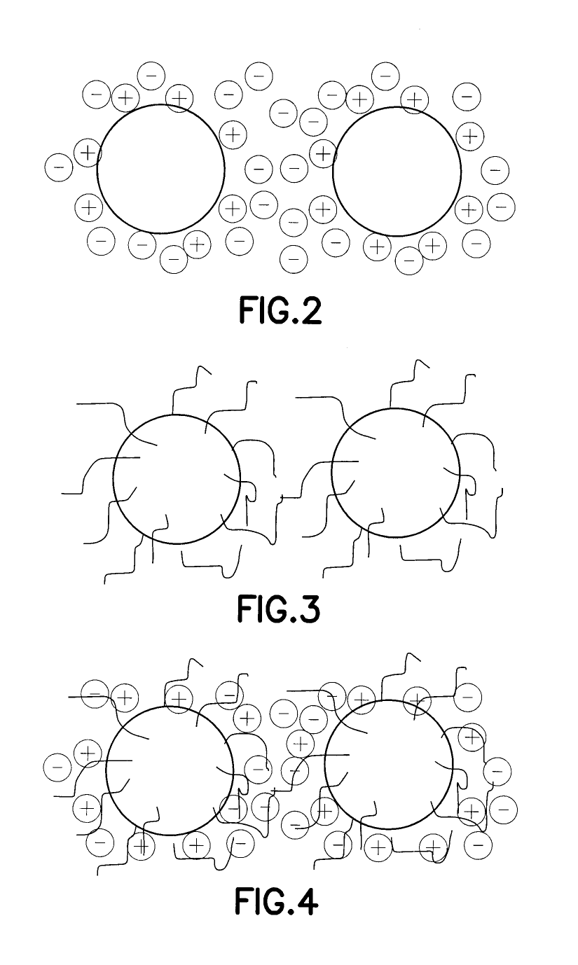

FIG. 2 to FIG. 4 disclose the three main ways to stabilize nanocolloids, namely: FIG. 2 shows a prior art schematic diagram of stabilized nanoparticles by electrostatic repulsion; FIG. 3 shows a prior art schematic diagram of stabilized nanoparticles by steric barrier; and FIG. 4 shows a prior art schematic diagram of stabilized nanoparticles by electrosteric interactions. The extra penalty energy added by these methods is electrostatic repulsion of particles with similar charges, steric forces form physical and the combination of both electrostatic and steric forces, respectively. The subject matter disclosed focuses on electrostatic stabilization (see FIG. 2). In FIG. 2, the electrostatic stabilization is prone to change the electrical double layer thickness because of agitation, temperature change and ionic species.

FIG. 5 shows the determination of the absorbance threshold wavelength of cadmium sulfide formed in methyldiethanolamine (MDEA) at 100° C., wherein the threshold wavelength is the wavelength were both black lines cross. A polar organic solvent may be used to suspend the metal sulfide that is insoluble in many solvents including water and most organic solvents. The electrostatic interaction between solvent and metal sulfide appears to have two important effects: (1) limiting the size of the particles formed; and (2) preventing precipitation/aggregation of the metal sulfide. This allows quantitative analysis of sulfide using absorption spectroscopy without interference from scattering.

Still referring to FIG. 5, many metals react with sulfide to form metal sulfides that absorb light at visible wavelengths (i.e., lead, copper, selenium, nickel, cobalt, cadmium and others) or in the ultra violet (UV) range (zinc and others). Absorbance in the visible range is preferable over absorbance in the UV range because many components will absorb in the UV range. Cadmium is of special interest because of its good solubility in many polar organic solvents, its high reactivity towards sulfide, the relative low density of cadmium sulfide, and the relative small maximum size of its quantum dots. Furthermore, cadmium has a preference to form cadmium sulfide over cadmium oxide. Cadmium sulfide is stable for many days exposed to air and even has a relatively good stability in slightly acidic solutions. The density difference that the electrostatic interaction between the nanoparticle and the solvent has to overcome is directly related to the density of the metal sulfide. A relative low density like cadmium sulfide (4.82 g/cm³) has, is therefore favorable above the higher density of lead sulfide (7.70 g/cm³). Furthermore, a slow Ostwald ripening process, small particles dissolve and redeposit onto larger particles, reduces the growth rate of particles after initial reaction and makes certain materials more suitable then others. Ostwald ripening for cadmium sulfide is much slower than for lead sulfide under similar conditions. The low density of cadmium sulfide (4.82 g/cm³) compared to other metal sulfides (lead sulfide is 7.70 g/cm³, nickel sulfide is 5.3 g/cm³) makes it less likely to precipitate. Quantum dots will have a threshold of the absorption peak that is dependent of its size, thus the absorption is not only dependent on the amount of metal sulfide but also on the size of the metals sulfide particles, making them particles in the quantum dot size less suitable for hydrogen sulfide detection.

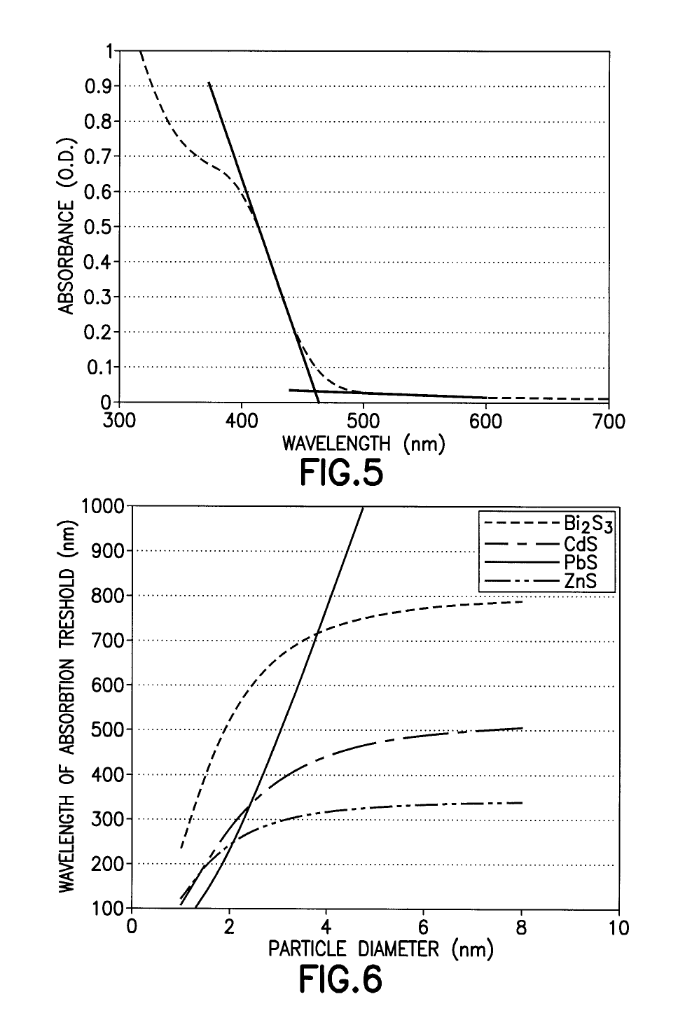

FIG. 6 shows a prior art theoretical threshold of absorbance for various metal sulfide particles according to the “effective mass model of quantum confinement,” as developed by Brus (L. E. Brus, J. Chem. Phys., vol. 80 (1984) 4403-4409). It is noted from the above remarks that a certain size threshold of the absorption is independent of the particle size and the particles are suitable for hydrogen sulfide detection.

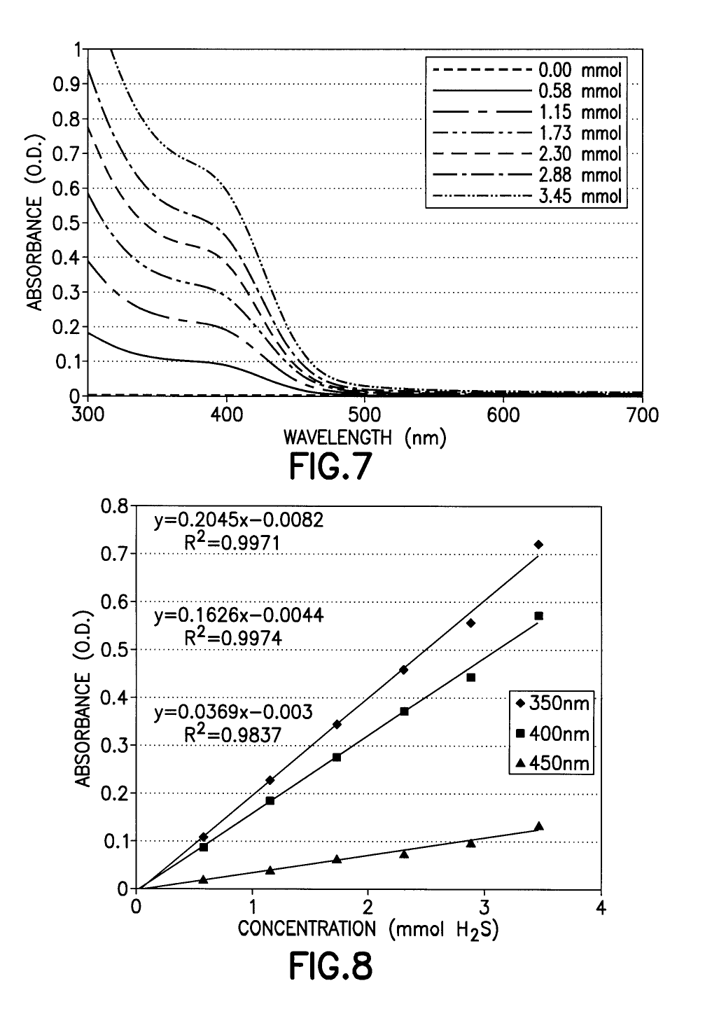

FIG. 7 shows the absorbance spectra of various concentrations of cadmium sulfide at room temperature. The cadmium sulfide is formed in a reaction of 4 mM cadmium sulfate in methyldiethanolamine (MDEA) with sulfide at 100° C.

Still referring to FIG. 7, the polar organic solvent is preferably of high density to give additional support to prevent precipitation. The solvent should have a polarity of at least 3 Debye units (D). An example of such a solvent is dimethyl sulfoxide (DMSO), which has good stabilizing properties at room temperature (20° C.). However, at higher temperatures the electrostatic interaction between DMSO and cadmium sulfide is not strong enough to prevent precipitation. It appears that the electrostatic interaction of amines with the cadmium and cadmium sulfide is stronger than between cadmium sulfide and DMSO. An example of a solvent that contains an amine and has a high density and a polarity above 3 Debye units (D) is methyldiethanolamine (MDEA). In particular, FIG. 7 shows the typical visible spectra of 4 mM cadmium sulfate in MDEA reacted with increasingly higher concentration of sulfide at 100° C. The curves exhibit a shoulder around 400 nm and have no peak.

FIG. 8 shows the absorbance as function of the cadmium sulfide concentration at various wavelengths: wherein the absorbance at a certain wavelength is the absorbance at this wavelength minus the absorbance at 550 nm; and the cadmium sulfide is formed in a reaction of 4 mM cadmium sulfate in methyldiethanolamine (MDEA) with sulfide at 100° C. Wherein, the sensitivity of the approach is better than 0.2 mM cadmium sulfide as shown in FIG. 8. It is note the proportional growth of the optical density with the sulfide concentration is the foundation for a quantitative measurement.

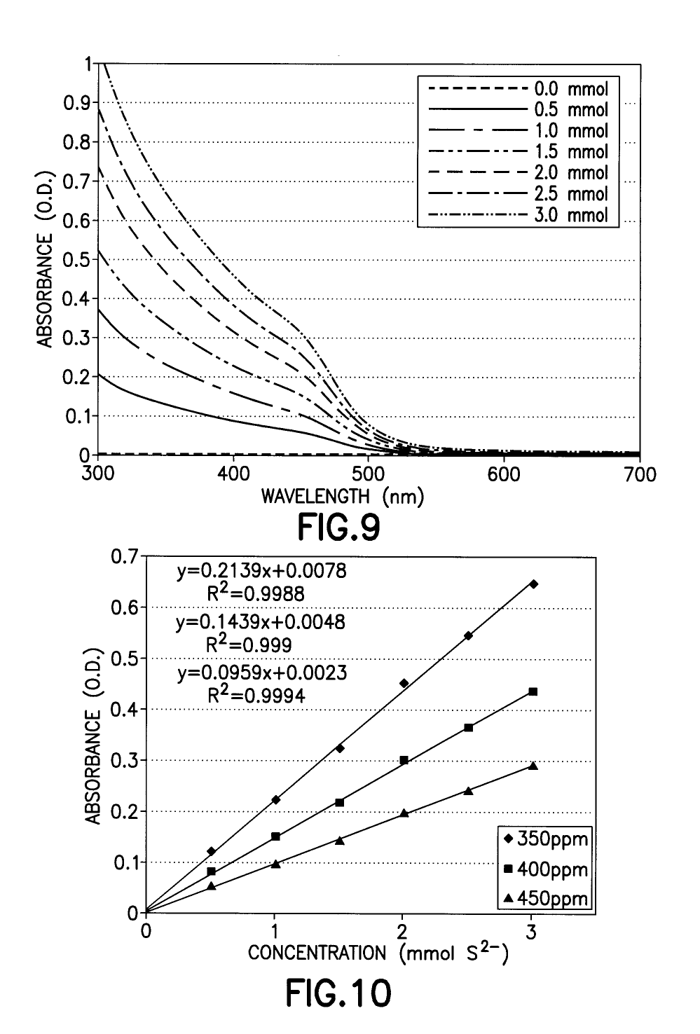

FIG. 9 shows the absorbance spectra of various concentrations of cadmium sulfide at room temperature, wherein the cadmium sulfide is formed in a reaction of 4 mM cadmium sulfate and 8 mM nitrilotriacetic acid (NTA) in triethanolamine (TEA) with sulfide at 150° C.

Still referring to FIG. 9, the threshold of the absorbance is slightly dependent on the reaction temperature. Reaction at a known preferably higher temperature will reduce the influence on the outcome of the measurement. Another method to reduce this influence and at the same time improve the stability of the metal ions and metal sulfide in the solution is the addition of a chelating agent. The chelating agent can be an amine, a sulfur containing compound or an polyalcohol. A good example of a chelating agent is nitrilotriacetic acid (NTA). Also shown in FIG. 9 is the absorption spectra of 4 mM cadmium sulfate and 8 mM NTA in triethanolamine (TEA) reacted with increasing concentrations of sulfide at 150° C. The curves exhibit a shoulder around 450 nm and have no peak.

FIG. 10 shows the absorbance as function of the cadmium sulfide concentration at various wavelengths. The absorbance at a certain wavelength is the absorbance at this wavelength minus the absorbance at 550 nm. The cadmium sulfide is formed in a reaction of 4 mM cadmium sulfate and 8 mM NTA in TEA with sulfide at 150° C.

Still referring to FIG. 10, the sensitivity of the approach is better than 0.2 mM cadmium sulfide. Also, it is note that the proportional growth of the optical density with the sulfide concentration is the foundation for a quantitative measurement.

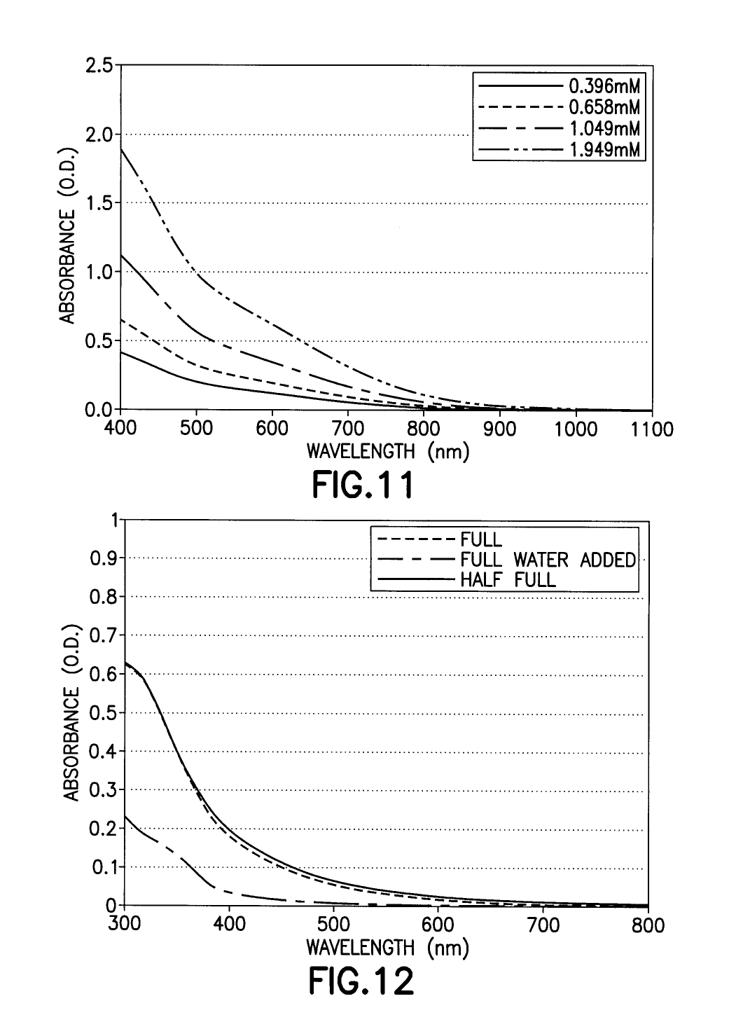

Referring to FIGS. 11 and 12, FIG. 11 shows the absorbance spectra of various concentrations of bismuth sulfide, wherein the bismuth sulfide is formed in a reaction of 2 mM bismuth nitrate with and 4 mM nitrilotriacetic acid (NTA) in triethanolamine (TEA) with sulfide at room temperature. Concentrations are expressed in mM sulfide. The chelating agent NTA is added to help dissolve the bismuth nitrate as well as to reduce the dependence of the particle size on the reaction temperature.

Still referring to FIGS. 11 and 12, the extended exposure (24 hrs.) of the solvents to elevated temperatures (above 100° C.) causes some coloring of the solvent (MDEA and TEA). This coloring will interfere with the absorbance measurements unless proper precautions are made. These precautions can be a baseline measurement directly before the reaction takes place. Preventing the coloring to take place is another approach to solve this problem. Addition of a small amount of water (e.g. 2.5% v/v) improved the thermal stability of the solution and reduced the coloring of the solution during extended periods (24 hrs.) of heating (150° C.). At least part of the coloring is due to the oxidation of the solvent by oxygen. Still referring to FIGS. 11 and 12, FIG. 12 shows absorbance curves of TEA after 25 hrs exposure to 150° C. Experiment performed in a closed vessel. Full means that the vessel is filled almost completely 42 ml triethanolamine (TEA) and 5 ml air, half full means 20 ml TEA and 27 ml air, full water added means 41 ml TEA, 1 ml 0.2 M Cd50₄ in water and 5 ml air.

Still referring to FIGS. 11 and 12, the absorbance spectra of MDEA and TEA after 25 hrs at 150° C. and the influence of air and the addition of cadmium sulfate in water on these absorption spectra.

It is possible the method can further comprise a radical scavenging agent to sustain endurance at high temperatures before and after reaction, and to functionalize the reagent mixture to exhibit properties outside the natural characteristics of the metal sulfide nanoparticles such as preventing coloration of the solvent upon heating and reducing a rate of degradation of the metal sulfide nanoparticles. The radical scavenging agent can be between about 0 to 5 volume percent.

Further, while the present disclosed subject matter has been described with reference to an exemplary embodiment, it is understood that the words, which have been used herein, are words of description and illustration, rather than words of limitation. Changes may be made, within the purview of the appended claims, as presently stated and as amended, without departing from the scope and spirit of the present disclosed subject matter in its aspects. Although the present disclosed subject matter has been described herein with reference to particular means, materials and embodiments, the present disclosed subject matter is not intended to be limited to the particulars disclosed herein; rather, the present disclosed subject matter extends to all functionally equivalent structures, methods and uses, such as are within the scope of the appended claims.

What is claimed is:

1. A reagent mixture for use in spectroscopic detection of hydrogen sulfide, the reagent mixture comprising:
metal ions dissolved in a fluid, the fluid is one of a ionic liquid or a polar organic solvent for reacting with a hydrogen sulfide sample thereby forming a metal sulfide nanoparticles, the polar organic solvent includes two or more properties having a density above 1 kg/l and a electrical dipole moment above 2 Debye units, wherein the ionic liquid and the polar organic solvent provide for stabilizing the metal sulfide nanoparticles and for preventing precipitation by electrostatic stabilization;
dissolving the metal ions into one of the ionic liquid or the polar organic solvent to create the reagent mixture;
mixing the reagent mixture with the hydrogen sulfide sample in a formation under downhole conditions at a temperature above 100 Celsius and at a pressure above 100 psi, wherein the metal ions of the reagent mixture react with the hydrogen sulfide sample to form the metal sulfide nanoparticles, resulting in the metal sulfide nanoparticles having an optical signature within a range from 200 nm to about 900 nm.

2. The reagent mixture according to claim 1, wherein the metal ions are from a soft metal group consisting of cadmium, mercury, silver, gold, palladium, rhodium, ruthenium, osmium, iridium, platinum or thallium.

3. The reagent mixture according to claim 1, wherein the metal ions are from the intermediate metal group consisting of manganese, iron, cobalt, nickel, copper, zinc, molybdenum, technetium, indium, tantalum, tungsten, rhenium, lead or bismuth.

4. The reagent mixture according to claim 1, further comprising a chelating ligand to dissolve the metal ions.

5. The reagent mixture according to claim 4, wherein a ratio of the chelating ligand to the metal ion is between about 0 to 2.

6. The reagent mixture according to claim 1, wherein the polar organic solvent is from an alkanolamines group including triethanolamine, methyldiethanolamine or some combination thereof.

7. The reagent mixture according to claim 1, wherein the polar organic solvent is dimethyl sulfoxide (DMSO).

8. The reagent mixture according to claim 1, wherein the spectroscopy is used for the detection of the hydrogen sulfide after the homogenous reagent mixture is exposed to hydrogen sulfide sample to form the metal sulfide nanoparticles.

9. The reagent mixture according to claim 1, wherein the metal ions are cadmium.

10. The reagent mixture according to claim 1, wherein detecting hydrogen sulfide in the formation fluid is under downhole conditions at sustained temperatures of one of 100 degree Celsius or more, 200 degree Celsius or more or 250 degree Celsius or more.

11. The reagent mixture according to claim 1, wherein the ionic liquid is from the group consisting of one of ammonium based, phosphonium based or imidazolium based cations.

12. The reagent mixture according to claim 1, wherein the ionic liquid contains a dication.

13. A method for use in spectroscopic detection of hydrogen sulfide in a formation fluid, the method comprising:
obtaining metal ions to be dissolved in a fluid, the fluid is one of a ionic liquid or a polar organic solvent for reacting with a hydrogen sulfide sample thereby forming a metal sulfide nanoparticles, the polar organic solvent includes two or more properties having a density above 1 kg/l and a electrical dipole moment above 2 Debye units, wherein the ionic liquid and the polar organic solvent provide for stabilizing the metal sulfide nanoparticles and for preventing precipitation by electrostatic stabilization
combining the metal ions into one of the ionic liquid or the polar organic solvent to create a homogenous reagent mixture;
mixing the homogenous reagent mixture with the hydrogen sulfide sample in the formation under downhole conditions at a temperature above 100 Celsius and at a pressure above 200 psi, wherein the metal ions of the homogenous reagent mixture react with the hydrogen sulfide to form the metal sulfide nanoparticles, resulting in the metal sulfide nanoparticles having an optical signature within a range from 200 nm to about 900 nm; and
spectroscopically interrogating the homogenous reagent mixture and formation fluid so as to detect the presence of the metal sulfide thereby indicating the presence of hydrogen sulfide in the formation fluid.

14. The method according to claim 13, wherein the spectroscopy interrogating includes measuring the optical property such as one of an optical density of the metal sulfide nanoparticles or a fluorescence of the metal sulfide nanoparticles, so a quantity of hydrogen sulfide in the formation fluid is capable of being measured while under downhole conditions.

15. The method according to claim 14, wherein the spectroscopy interrogating includes before mixing, measuring the optical property such as the optical density or the fluorescence of one of the homogenous reagent mixture, the formation fluid or both.

16. The method according to claim 14, wherein the combining further comprises introducing the homogenous reagent mixture into a downhole flowline containing the formation fluid and the spectroscopically interrogating further comprises interrogating through an optical window in the flowline downstream from the location of introduction of the detection mixture.

17. The method according to claim 14, wherein the combining further comprises introducing the formation fluid into a container containing the homogenous reagent mixture, and the interrogating further comprises interrogating through an optical window in a wall of the container while in a subterranean environment.

18. The method according to claim 17, wherein the combining further comprises mechanically stifling the homogenous reagent mixture and the formation fluid in the container to shorten a rate of time used to carry out the interrogating.

19. The method according to claim 14, wherein the combining further comprises introducing the formation fluid into a container containing the homogenous reagent mixture, and introducing the combined homogenous reagent mixture and the formation fluid from the container into a downhole flowline, and spectroscopically interrogating through an optical window in a wall of the flowline.

20. The method according to claim 14, further comprising adding chelating ligands to the homogenous reagent mixture for sustaining thermal endurance of the homogenous reagent mixture under downhole conditions, wherein the metal ions are cadmium, and the spectroscopically interrogating is through an optical window in a wall of the flowline while in a subterranean environment.

21. The method according to claim 14, wherein the method of detecting hydrogen sulfide in the formation fluid is under downhole conditions is at sustained temperatures of one of 100 degree Celsius or more, 200 degree Celsius or more or 250 degree Celsius or more.

22. The method according to claim 14, wherein spectroscopically interrogating of the homogenous reagent mixture and formation fluid is completed on a surface of the earth.

23. A reagent mixture for use in spectroscopic detection of hydrogen sulfide, the reagent mixture comprising:
metal ions dissolved in a ionic liquid for reacting with a hydrogen sulfide sample thereby forming a metal sulfide nanoparticles, wherein the ionic liquid includes at least one positive ion and at least one negative ion, that provides stabilizing the metal sulfide nanoparticles and for preventing precipitation by electrostatic stabilization;
dissolving the metal ions into the ionic liquid to create the reagent mixture;
mixing the reagent mixture with the hydrogen sulfide sample in a formation under downhole conditions at a temperature above 150 Celsius and at a pressure above 150 psi,
wherein the metal ions of the reagent mixture react with the hydrogen sulfide sample to form the metal sulfide nanoparticles, resulting in the metal sulfide nanoparticles having properties with an optical signature within a range from 200 nm to about 900 nm and the metal sulfide nanoparticles is a nanoparticle kept in suspension to form a homogenous reagent mixture.
